(19) United States
(12) Patent Application Publication
(10) Pub. No.: US 2026/0196067 A1
(43) Pub. Date: Jul. 9, 2026

(54) Gene Image Data Correction Method and System, Electronic Device, and Storage Medium

(71) Applicants: BGI SHENZHEN, Shenzhen (CN); BGI SHENZHEN CO., LTD, Shenzhen (CN)

(72) Inventors: Yiwen Wu, Shenzhen (CN); Min Li, Shenzhen (CN); Mei Li, Shenzhen (CN); Yuxiang Li, Shenzhen (CN)

(21) Appl. No.: 19/135,613

(22) PCT Filed: Dec. 5, 2022
(86) PCT No.: PCT/CN2022/136670

§ 371 Date: Jun. 4, 2025

(51) Int. Cl. G06V 20/69; G06T 7/13; G06T 7/30; G06V 10/26; G16B 25/00

(52) U.S. Cl. CPC G06V 20/698; G06T 7/13; G06T 7/30; G06V 10/26; G06V 20/695; G16B 25/00; G06T 2207/10056; G06T 2207/30024; G06T 2207/30242; G06V 2201/03

(57) ABSTRACT

A gene image data correction method and system, an electronic device, and a storage medium are disclosed. The gene image data correction method includes determining a gene molecule density of a target cell based on a count and an initial contour area; determining a background gene molecule density based on a count and a region area of background gene molecules; and determining a cell to which the background gene molecules belong based on the gene molecule density and the background gene molecule density.

### CROSS-REFERENCE TO RELATED APPLICATION

This application is the United States national phase of International Patent Application No. PCT/CN2022/136670 filed Dec. 5, 2022, the disclosure of which is hereby incorporated by reference in its entirety.

### BACKGROUND

### Technical Field

The present disclosure relates to the technical field of gene image processing, and in particular to a gene image data correction method and system, an electronic device, and a storage medium.

### Technical Considerations

RNA cell labeling (a technique for labeling genetic information within living cells) involves assigning genes to cells based on registered cell contour images and gene expression data corresponding to the cells. However, due to challenges such as insufficient intracellular gene counts, nuclear segmentation issues, and RNA (ribonucleic acid, the genetic information carrier in biological cells and certain viruses/viroids) diffusion artifacts, the number of genes assigned to a cell is significantly lower than the actual genes contained within the cell. This necessitates correction of RNA cell labeling results to improve accuracy of RNA cell labeling. RNA cell labeling is a critical step in the downstream analysis of Stereo-seq (Spatio-Temporal Enhanced Resolution Omics-sequencing) spatial transcriptomics and serves as the foundation for other subsequent analyses. Current correction algorithms for RNA cell labeling rely on cell morphology derived from cell contour images, exhibiting weak robustness and low accuracy, with a substantial proportion of RNA molecules remaining unclassified. Significant opportunities exist for further exploration and optimization of this issue.

### SUMMARY

A main objective of the present disclosure is to provide a gene image data correction method and system, an electronic device, and a storage medium, aiming at addressing defects in the existing technology such as over-reliance on cell morphology from cell contour images and failure to classify a significant number of RNA molecules.

The present disclosure addresses the aforementioned technical problem through the following technical schemes.

In accordance with a non-limiting aspect of the present disclosure, there is provided a gene image data correction method, including:
determining an initial count of gene molecules belonging to a target cell in a gene image and an initial contour area of a target region occupied by the gene molecules;
determining a gene molecule density of the target cell based on the initial count and the initial contour area;
determining a count of background gene molecules within a preset region of the target region and a region area of the preset region;
determining a background gene molecule density based on the count of the background gene molecules and the region area; and
determining a cell to which the background gene molecules belong based on the gene molecule density and the background gene molecule density.

In non-limiting embodiments, determining a cell to which the background gene molecules belong based on the gene molecule density and the background gene molecule density includes:
in response to a difference between the gene molecule density and the background gene molecule density being within a predetermined range, correcting the background gene molecules as gene molecules belonging to the target cell.

In non-limiting embodiments, determining a background gene molecule density based on the count of the background gene molecules and the predetermined area includes:
determining a plurality of preset regions of different sizes; and
determining background gene molecule densities corresponding to the plurality of preset regions, respectively; and
correcting the background gene molecules as gene molecules belonging to the target cell includes:
   correcting background gene molecules within a preset region as gene molecules belonging to the target cell, where the background gene molecules within the preset region exhibit the smallest difference in gene molecule density compared to the target cell.
In non-limiting embodiments, determining an initial count of gene molecules belonging to a target cell in a gene image includes:
acquiring a microscope image and a gene image of a biological sample;
performing image registration on the microscope image and the gene image of the biological sample;
performing cell segmentation on the microscope image to obtain a cell segmentation result;
determining a gene image of a target cell based on the cell segmentation result and an image registration result of the gene image; and
determining an initial count of gene molecules belonging to the target cell based on the gene image of the target cell.

In non-limiting embodiments, determining an initial contour area of a target region occupied by the gene molecules belonging to the target cell includes:
calculating, by using a convex hull algorithm, to determine an initial contour area of a target region occupied by the gene molecules belonging to the target cell based on the cell segmentation result.

In non-limiting embodiments, determining a gene molecule density of the target cell based on the initial count and the initial contour area includes:
calculating a ratio of the initial count to the initial contour area to determine the gene molecule density of the target cell.

In non-limiting embodiments, determining a background gene molecule density based on the count of the background gene molecules and the region area includes:
calculating a ratio of the molecule of the background gene molecules to the region area of the background gene molecules to determine the background gene molecule density.

In non-limiting embodiments, after determining a cell to which the background gene molecules belong based on the gene molecule density and the background gene molecule density, the method further includes:
generating gene expression information of the target cell based on a corrected result of gene molecules belonging to the target cell.

In accordance with a non-limiting aspect of the present disclosure, there is provided a gene image data correction system, including:
at least one processor, comprising:
a determination module configured to determine an initial count of gene molecules belonging to a target cell in a gene image and an initial contour area of a target region occupied by the gene molecules; determine a gene molecule density of the target cell based on the initial count and the initial contour area; determine a count of background gene molecules within a preset region of the target region and a region area of the preset region; and determine a background gene molecule density based on the count of the background gene molecules and the region area; and
a correction module configured to determine a cell to which the background gene molecules belong based on the gene molecule density and the background gene molecule density.

In accordance with a non-limiting aspect of the present disclosure, there is provided an electronic device including a memory, at least one processor, and a computer program stored in the memory and executable by the processor, where the computer program, when executed by the at least one processor, causes the at least one processor to implement the gene image data correction method as described above.

In accordance with a non-limiting aspect of the present disclosure, there is provided a computer-readable medium storing computer instructions that, when executed by at least one processor, cause the at least one processor to implement the gene image data correction method as described above.

The positive progress effect of the present disclosure lies in the following.

Because in practice, gene molecules are uniformly distributed within the actual contour of a target cell, the gene molecule density within the initial contour of the target cell should be comparable to the density of background gene molecules, where the background gene molecules are outside the initial contour of the target cell but within the actual contour of the target cell. The present disclosure leverages the above-mentioned cellular characteristic, where the gene molecule density of the target cell is determined based on the initial count of gene molecules belonging to the target cell and the initial contour area of the target cell in the gene image. The gene molecule density of the target cell is compared with the background gene molecule density, and background gene molecules exhibiting a difference between the two within a predetermined range are classified as gene molecules of the target cell. This significantly improvs the efficiency of correcting gene molecules within a target cell while also increasing the accuracy of the correction.

### BRIEF DESCRIPTION OF THE DRAWINGS

FIG. 1a is a distribution map of gene molecules before correction using a spatiotemporal data correction algorithm;

FIG. 1b is a distribution map of gene molecules after correction using the spatiotemporal data correction algorithm;

FIG. 2 is a first flowchart of a non-limiting embodiment of a gene image data correction method according to the present disclosure;

FIG. 3 is a second flowchart of the non-limiting embodiment of the gene image data correction method;

FIG. 4 is a schematic diagram of centroids of target cells in the non-limiting embodiment of the gene image data correction method;

FIG. 5 is a third flowchart of the non-limiting embodiment of the gene image data correction method;

FIG. 6 is a fourth flowchart of the non-limiting embodiment of the gene image data correction method;

FIG. 7a is a visualization of a first spatial clustering of a mouse brain before correction using the non-limiting embodiment of the gene image data correction method;

FIG. 7b is a visualization of a second spatial clustering of a mouse brain before correction using the non-limiting embodiment of the gene image data correction method;

FIG. 7c is a visualization of a third spatial clustering of a mouse brain before correction using the non-limiting embodiment of the gene image data correction method;

FIG. 7d is a visualization of the first spatial clustering of a mouse brain after correction using the non-limiting embodiment of the gene image data correction method;

FIG. 7e is a visualization of the second spatial clustering of a mouse brain after correction using the non-limiting embodiment of the gene image data correction method;

FIG. 7f is a visualization of the third spatial clustering of a mouse brain after correction using the first non-limiting embodiment of the gene image data correction method;

FIG. 8a is a visualization of target cells obtained with the UMAP algorithm (a dimensionality reduction algorithm) and the Leiden algorithm (a clustering algorithm) before correction using the non-limiting embodiment of the gene image data correction method;

FIG. 8b is a visualization of target cells obtained with the UMAP algorithm and the Leiden algorithm after correction using the non-limiting embodiment of the gene image data correction method;

FIG. 9 is a diagram of morphological changes of a plurality of target cells after correction using the first non-limiting embodiment of the gene image data correction method;

FIG. 10 is a first block diagram of a non-limiting embodiment of a gene image data according to the principles of the present disclosure;

FIG. 11 is a second block diagram of a non-limiting embodiment of a gene image data correction system; and

FIG. 12 is a schematic diagram of a non-limiting embodiment of an electronic device according to the principles of the present disclosure.

### DETAILED DESCRIPTION

The following further illustrates the present disclosure through embodiments, but does not limit the present disclosure to the scope of the described embodiments.

### Gene Image Data Correction Method

In the process of high-throughput sequencing analysis, by registering Stereo-seq spatial transcriptome data with single-stranded DNA (ssDNA) images, and performing cell segmentation on the ssDNA images, correspondence between pixel coordinates and cells is established. The Stereo-seq spatial transcriptome data is converted into gene expression data of the cells through this correspondence for downstream analysis.

However, since the cell size obtained through ssDNA image-based segmentation only reflects nuclear dimensions, resulting in discrepancies from actual cell size, along with issues including insufficient gene counts per cell in sequencing experiments, nuclear segmentation artifacts, and RNA diffusion effects, the genes assigned to cells are far fewer than those actually present in the cells. This leads to low data accuracy throughout the sequencing process, introduces errors in gene expression data, and consequently reduces sequencing efficiency.

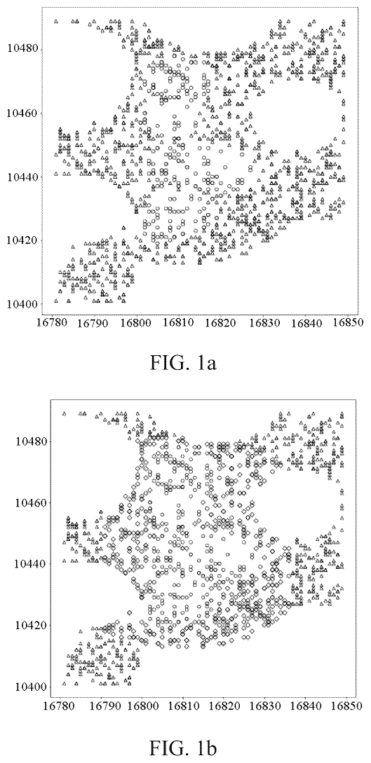

A spatiotemporal data correction algorithm based on Gaussian Mixture Model (GMM) is implemented to adjust RNA cell labeling results. Specifically, the algorithm uses GMM to calculate probability scores for gene molecules belonging to neighboring cells. GMM parameters are derived through fitting based on the RNA distribution within the current cell (primarily based on ssDNA spatial coordinates and unique molecular identifier (UMI) values). Using the GMM parameters derived through fitting, the probability scores of extracellular gene molecules near the current cell boundary are calculated. With reference to FIG. 1a, the probabilities of gene molecules in the background are calculated (the triangles in FIG. 1a represent gene molecules in the background, and the dots in FIG. 1a represent the original results of RNA cell labeling). A probability threshold filtering criterion is applied, such that RNA gene molecules that meet the criterion are categorized into cell interiors (the quadrilaterals in FIG. 1b represent gene molecules that have been reclassified as belonging to the current cell after correction), while those that do not meet the criterion remain as extracellular background (the triangles in FIG. 1b represent gene molecules that still reside in the background after correction). However, the spatiotemporal data correction algorithm relies on the cell morphology derived from cell contour images, exhibiting weak robustness and low accuracy, with a substantial number of gene molecules remaining unclassified.

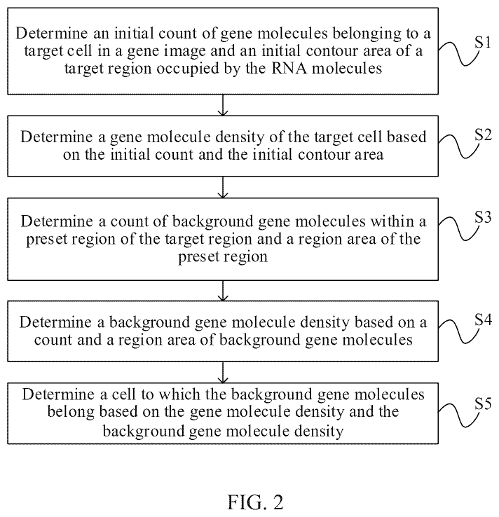

In order to overcome the aforementioned defects currently present, this non-limiting embodiment provides a gene image data correction method, which is implemented by or through at least one processor. As shown in FIG. 2, the gene image data correction method includes the following steps.

At S1, an initial count of gene molecules belonging to a target cell in a gene image and an initial contour area of a target region occupied by the gene molecules are determined.

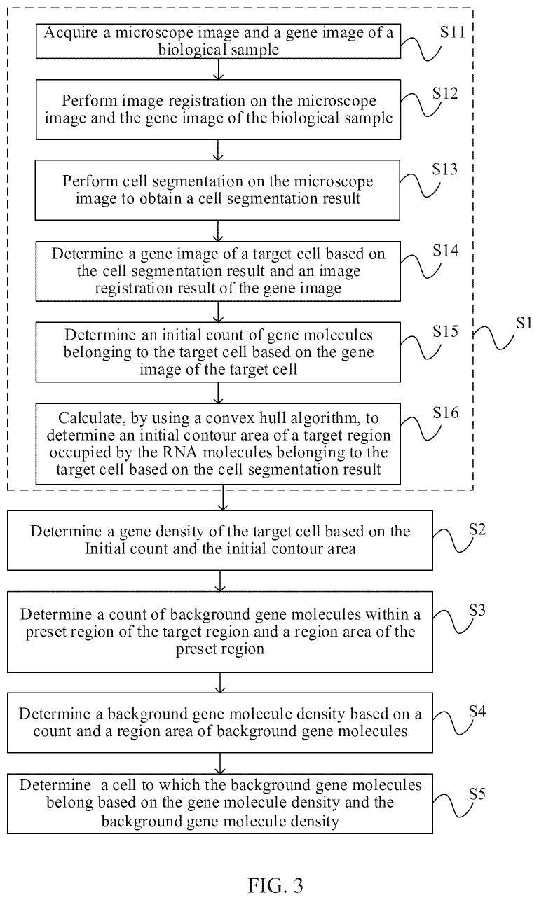

In an alternative implementation, as shown in FIG. 3, the step of determining an initial count of gene molecules belonging to a target cell in a gene image mainly includes the following sub-steps.

At S11, a microscope image and a gene image of a biological sample are acquired.

Herein, the microscope image and the gene image include a plurality of target cells.

The microscope image may be an image obtained by photographing the biological sample through a microscope and it includes spatial information of the biological sample. The gene image includes spatial information and genetic data of the biological sample. Herein, the pixels in the gene image correspond to the elements of the gene matrix, and the pixel values correspond to the values of these elements. Therefore, the gene image can include the spatial information of the same biological sample.

In this embodiment, the microscope image may be represented as ssDNA image, without the intention to limit the type of the microscope image, and adjustments and selections can be made according to actual needs. The gene image may be represented as Stereo-seq spatial transcriptome data, or gene expression data, etc.

At S12, image registration is performed on the microscope image and the gene image of the biological sample.

For example, image registration is performed on the ssDNA image (i.e., microscope image) and the Stereo-seq spatial transcriptome data (i.e., gene image) to generate gene expression information of the cell.

In the process of image registration, the microscope image is rotated and/or scaled based on its markings. A preliminary offset, calculated from the centroids of the microscope image and the gene image, is then applied to preliminary registration of the microscope image with the gene image. The microscope image is corrected for offset based on the respective markings of the microscope image and the gene image. Subsequently, image registration is performed between the gene image and the corrected microscope image.

At S13, cell segmentation is performed on the microscope image to obtain a cell segmentation result.

That is, cell segmentation is performed on the microscope image to acquire the correspondence between spatial positions and cells, and based on this correspondence, a cell segmentation result is generated.

At S14, a gene image of a target cell is determined based on the cell segmentation result and an image registration result of the gene image.

Based on the gene image of the target cell, gene molecules can be classified as gene molecules belonging to the target cell and background gene molecules of unknown associated cells.

At S15, an initial count of gene molecules belonging to the target cell is determined based on the gene image of the target cell.

In an alternative implementation, as shown in FIG. 3, the step of determining an initial contour area of a target region occupied by the gene molecules belonging to the target cell includes the following step.

At S16, calculation using a convex hull algorithm is performed based on the cell segmentation result to determine an initial contour area of a target region occupied by the gene molecules belonging to the target cell.

Herein, the Convex Hull Algorithm involves finding the convex hull of N points within the target region based on the cell segmentation result, where the connections between these points enclose the initial contour area.

At S2, a gene molecule density of the target cell is determined based on the initial count and the initial contour area.

In an alternative implementation, a ratio of the initial count to the initial contour area is calculated to determine the gene molecule density of the target cell.

At S3, a count of background gene molecules within a preset region of the target region and a region area of the preset region are determined.

Herein, the background gene molecules are gene molecules that cannot be determined as belonging to a specific cell based on the gene image. The preset region is located outside the target region but must not go beyond regions occupied by gene molecules belonging to neighboring cells. A region with an area of 5 pixels*5 pixels may be selected as the preset region.

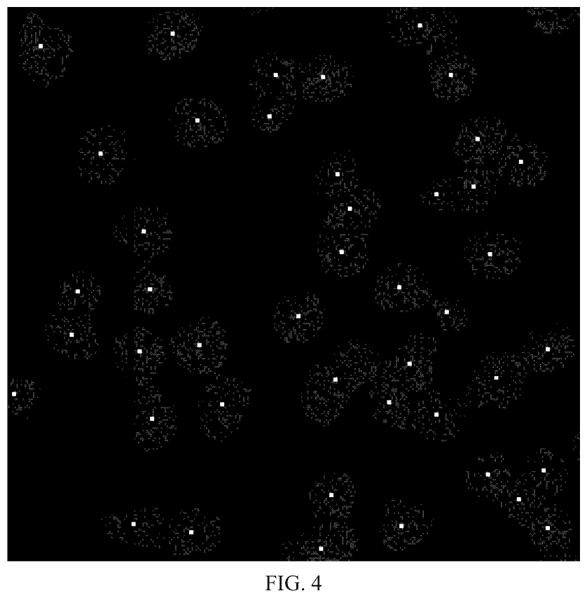

In an alternative implementation, the preset region can be defined as an area centered around the centroid of the target cell. The centroids are determined based on the gene image, and a schematic diagram of the centroids are shown in FIG. 4.

At S4, a background gene molecule density is determined based on a count and a region area of background gene molecules.

In an alternative implementation, the background gene molecule density is calculated as the ratio of the count of the background gene molecules to the region area of the background gene molecules.

At S5, a cell to which the background gene molecules belong is determined based on the gene molecule density and the background gene molecule density.

In this embodiment, the above-mentioned cellular characteristic is leveraged, where the gene molecule density of the target cell is determined based on the initial count of gene molecules belonging to the target cell and the initial contour area of the target cell in the gene image. The background gene molecule density is compared against the gene molecule density of the target cell, and the cell to which the background gene molecules belong is determined based on the gene molecule density and the background gene molecule density, thereby significantly improving the efficiency of correcting gene molecules within a target cell and also increasing the accuracy of the correction.

In an alternative implementation, step S5 includes the following sub-steps.

At S51, in response to a difference between the gene molecule density and the background gene molecule density being within a predetermined range, the background gene molecules are corrected as gene molecules belonging to the target cell.

In response to the difference between the gene molecule density and the background gene molecule density being not within the predetermined range, it is determined that the background gene molecules are not gene molecules of the target cell.

Because in practice, gene molecules are uniformly distributed within the actual contour of a target cell, the gene molecule density within the initial contour of the target cell should be comparable to the density of background gene molecules, where the background gene molecules are outside the initial contour of the target cell but within the actual contour of the target cell.

In this embodiment, the above-mentioned cellular characteristic is leveraged, where the gene molecule density of the target cell is determined based on the initial count of gene molecules belonging to the target cell and the initial contour area of the target cell in the gene image. The gene molecule density of the target cell is compared with the background gene molecule density, and background gene molecules exhibiting a difference between the two within a predetermined range are classified as gene molecules of the target cell. This significantly improves the efficiency of correcting gene molecules within a target cell while also increasing the accuracy of the correction.

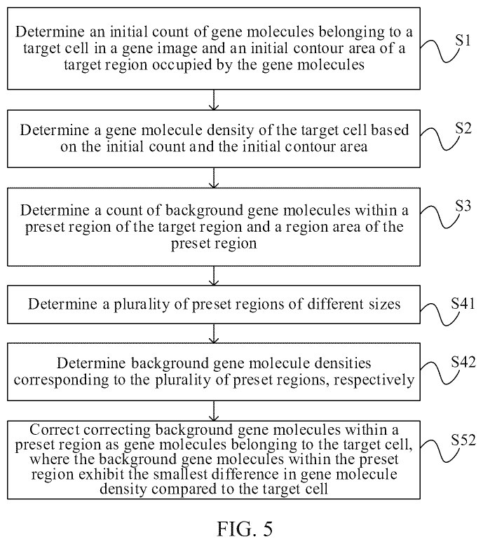

In an alternative implementation, with reference to FIG. 5, step S4 includes the following sub-steps.

At S41, a plurality of preset regions of different sizes are determined.

At S42, background gene molecule densities are determined corresponding to the plurality of preset regions, respectively.

with reference to FIG. 5, step S5 includes the following sub-steps.

At S52, background gene molecules within a preset region, exhibiting the smallest difference in gene molecule density compared to the target cell, are corrected as gene molecules belonging to the target cell.

In this implementation, by correcting background gene molecules within a preset region as gene molecules belonging to the target cell, where the background gene molecules within the preset region exhibit the smallest difference in gene molecule density compared to the target cell, the accuracy of correction of gene molecules within the target cell is further enhanced.

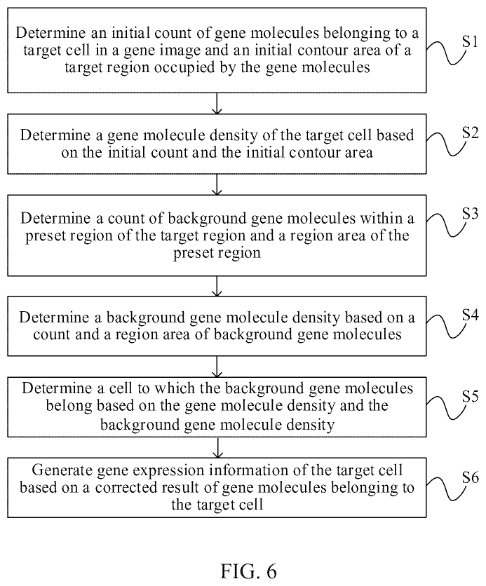

In an alternative implementation, with reference to FIG. 6, after step S5, the method includes the following step.

At S6, gene expression information of the target cell is generated based on a corrected result of gene molecules belonging to the target cell.

The following sets of experimental visualizations illustrate the beneficial effects of the gene image data correction method of this embodiment.

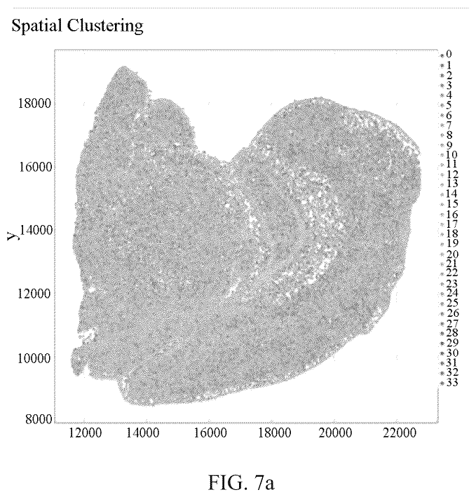

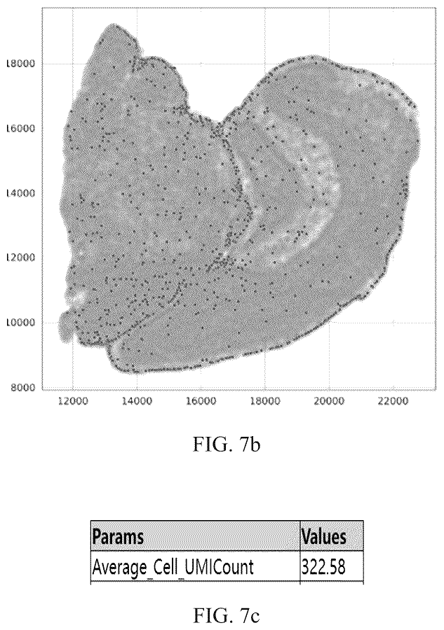

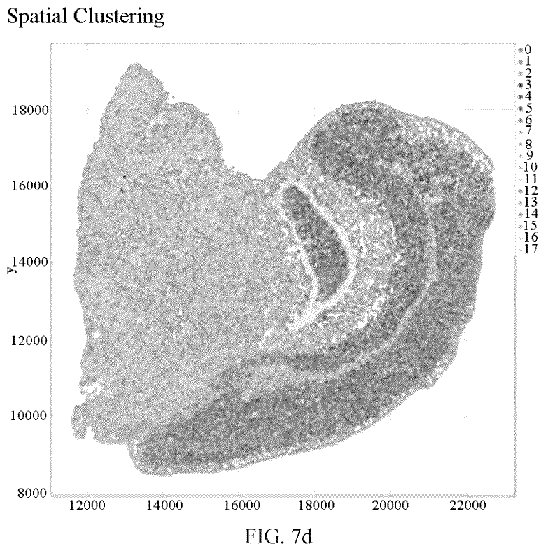

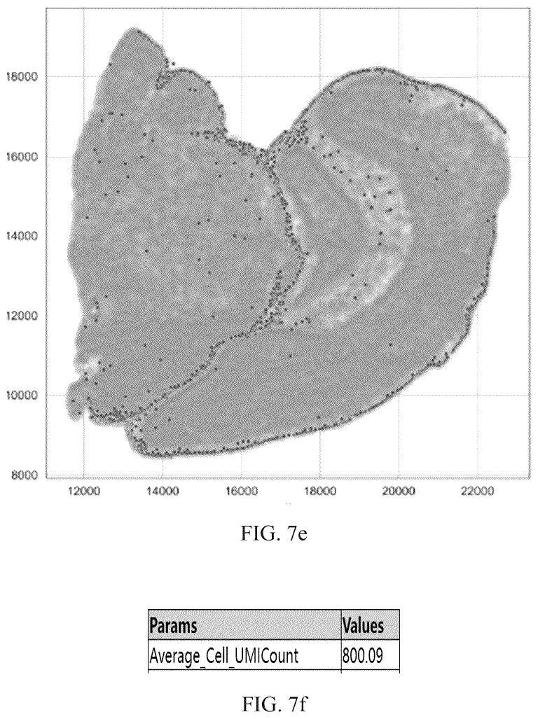

FIGS. 7a, 7b, and 7c show visualizations of spatial clustering of a mouse brain before correction. FIGS. 7d, 7e, and 7f show visualizations of the clustering of the mouse brain after correction. By comparison, it can be seen that the spatial positions of the corrected cell clusters are clearer, there are fewer intra-class noise points, and the average gene count per cell (Average_Cell_UMI Count) has increased, that is, the values corresponding to the parameters (Params) have increased. The units of the horizontal and vertical axes of FIGS. 7a, 7b, 7c, 7d, 7e, and 7f are all in pixels.

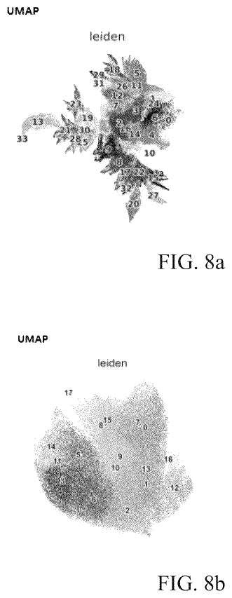

FIG. 8a shows a visualization with application of the UMAP algorithm (a dimensionality reduction algorithm) and the Leiden algorithm (a clustering algorithm) before correction; and FIG. 8b shows a visualization with application of the UMAP algorithm (a dimensionality reduction algorithm) and the Leiden algorithm (a clustering algorithm) after correction. Compared to FIG. 8a, the boundaries of different classes in FIG. 8b are clearer, resulting in better classification performance.

FIG. 9 shows the morphological changes of several target cells after correction, with black representing the newly added gene molecules belonging to the target cells.

### Gene Image Data Correction System

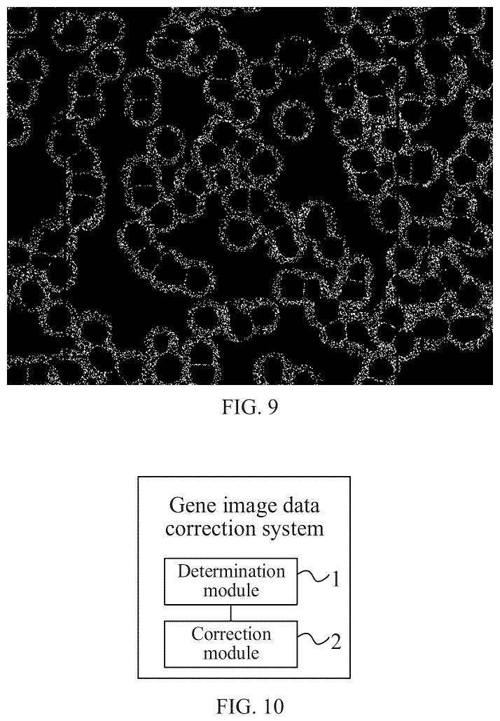

FIG. 10 shows a non-limiting embodiment of a gene image data correction system including at least one processor, including:
at least one processor, comprising:
a determination module 1 configured to determine an initial count of gene molecules belonging to a target cell in a gene image and an initial contour area of a target region occupied by the gene molecules; determine a gene molecule density of the target cell based on the initial count and the initial contour area; determine a count of background gene molecules within a preset region of the target region and a region area of the preset region; and determine a background gene molecule density based on the count of the background gene molecules and the region area; and
a correction module 2, configured to determine a cell to which the background gene molecules belong based on the gene molecule density and the background gene molecule density.

In an alternative implementation, the correction module 2 is further configured to correct the background gene molecules as gene molecules belonging to the target cell in response to a difference between the gene molecule density and the background gene molecule density being within a predetermined range. In an alternative implementation, the determination module 1 is further configured to determine a plurality of preset regions of different sizes; and further configured to determine background gene molecule densities corresponding to the plurality of preset regions, respectively.

The correction module 2 is further configured to correct background gene molecules within a preset region as gene molecules belonging to the target cell, where the background gene molecules within the preset region exhibit the smallest difference in gene molecule density compared to the target cell.

In an alternative implementation, as shown in FIG. 11, the gene image data correction system further includes:
an acquisition module 3 configured to acquire a microscope image and a gene image of a biological sample; and
a registration module 4 configured to perform image registration on the microscope image and the gene image of the biological sample.

The acquisition module 3 is further configured to perform cell segmentation on the microscope image to obtain a cell segmentation result; and

The determination module 1 is further configured to determine a gene image of a target cell based on the cell segmentation result and an image registration result of the gene image, and further configured to determine an initial count of gene molecules belonging to the target cell based on the gene image of the target cell.

In an alternative implementation, the determination module 1 is further configured to calculate, by using a convex hull algorithm, to determine an initial contour area of a target region occupied by the gene molecules belonging to the target cell based on the cell segmentation result.

In an alternative implementation, the determination module 1 is further configured to determine a ratio of the initial count and the initial contour area as the gene molecule density of the target cell.

In an alternative implementation, the determination module 1 is further configured to calculate a ratio of the molecule of the background gene molecules to the region area of the background gene molecules to determine the background gene molecule density.

In an alternative implementation, as shown in FIG. 11, the gene image data correction system further includes:
a generation module 5 configured to generate gene expression information of the target cell based on a corrected result of gene molecules belonging to the target cell.

It should be noted that the implementation principles and technical effects of the various modules in this embodiment can be found in the corresponding sections of Embodiment One, and therefore, details are not repeated herein.

### Electronic Device for Gene Image Data Correction

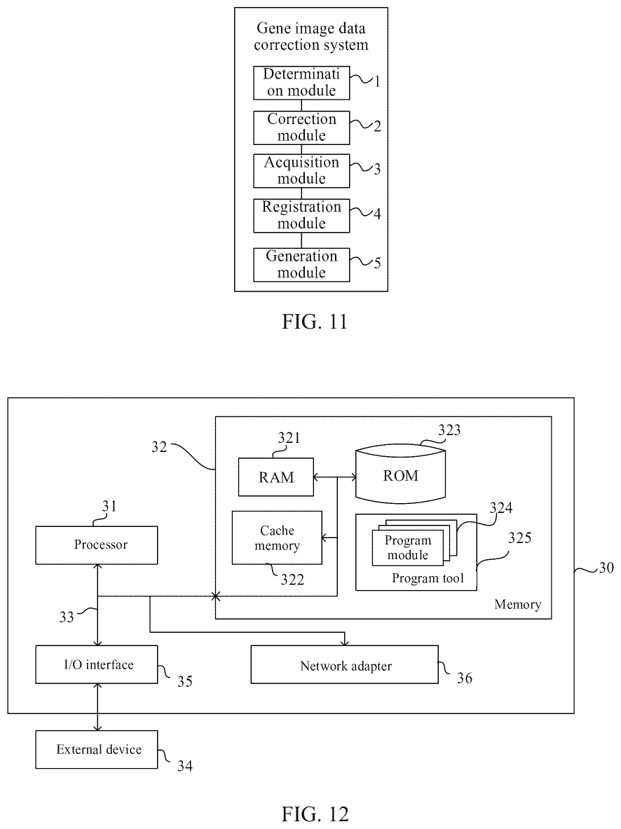

This non-limiting embodiment provides an electronic device. FIG. 12 is a schematic diagram of modules of the electronic device. The electronic device includes a memory, at least one processor, and a computer program stored in the memory and executable by the at least one processor, where the computer program, when executed by the at least one processor, causes the at least one processor to implement the gene image data correction method. The electronic device 30 shown in FIG. 12 is just an example and should not limit the scope of functions and application of the embodiments of the present disclosure.

As shown in FIG. 12, the electronic device 30 may be embodied in the form of a general-purpose computing device, for example, it can be a server device. Components of the electronic device 30 may include but are not limited to: at least one above-mentioned processor 31, at least one above-mentioned memory 32, and a bus 33 connecting different components (including the memory 32 and the processor 31).

The bus 33 includes a data bus, an address bus, and a control bus.

The memory 32 may include a volatile memory, such as a random access memory (RAM) 321 and/or a cache memory 322, and may further include a read-only memory (ROM) 323.

The memory 32 may also include a program/utility tool 325 with a set of (at least one) program module(s) 324, such program module 324 include but is not limited to: an operation system, one or more application programs, other program modules and program data, and each or some combinations of these examples may include the implementation of a network environment. The processor 31 executes various functional applications and data processing by running the computer program stored in the memory 32, such as the gene image data correction method.

The electronic device 30 may also communicate with one or more external devices 34 (such as a keyboard, a pointing device, etc.). Such communication can be done through an input/output (I/O) interface 35. Additionally, the model-generated device 30 may also communicate with one or more networks (such as local area networks (LAN), wide area networks (WAN), and/or public networks, such as the Internet) via a network adapter 36. As shown in FIG. 12, the network adapter 36 communicates with other modules of the model-generated device 30 via a bus 33. It should be understood that although not shown in the figure, other hardware and/or software modules may be used in conjunction with the model-generated device 30, including but not limited to: microcode, device drivers, redundant processors, external disk drive arrays, Redundant Array of Independent Disks (RAID) systems, tape drives, and data backup storage systems.

It should be noted that although several units/modules or sub-units/modules of the electronic device have been mentioned in the detailed description above, this division is merely exemplary and not mandatory. In practice, according to the implementation of the present disclosure, the features and functions of two or more units/modules described above may be embodied in a single unit/module. Conversely, the features and functions of a single unit/module described above can be further divided into multiple units/modules.

In another non-limiting embodiment, provided is a computer-readable storage medium storing a computer program that, when executed by at least one processor, causes the at least one processor to implement the gene image data correction method.

Herein, the computer-readable storage medium may include but is not limited to: a portable disk, a hard disk, a random access memory, a read-only memory, an erasable programmable read-only memory, an optical storage devices, a magnetic storage device, or any suitable combinations thereof.

In a possible implementation, the present disclosure may also be realized in the form of a program product, which includes program code that, when executed on a terminal device, causes the terminal device to execute the gene image data correction method of Embodiment One.

Herein, the program code for executing the present disclosure can be written in any combination of one or more programming languages, and the program code can be executed entirely on a user device, partially on a user device, as a standalone software package, partially on a user device and partially on a remote device, or entirely on a remote device.

In some non-limiting embodiments, determination module 1, correction module 2, acquisition module 3, registration module 4, generation module 5, and/or processor 31 may be implemented in hardware, firmware, or a combination of hardware and software. Non-limiting embodiments or aspects described herein are not limited to any specific combination of hardware circuitry (e.g., a processor) and/or software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

Although the specific embodiments of the present disclosure have been described above, those of ordinary skill in the art should understand that these are merely illustrative and non-limiting examples, and various changes or modifications may be made without departing from the principles and essence of the present disclosure. Therefore, the scope of protection of the present disclosure is defined by the appended claims.

1. A gene image data correction method, comprising:
determining an initial count of gene molecules belonging to a target cell in a gene image and an initial contour area of a target region occupied by the gene molecules;
determining a gene molecule density of the target cell based on the initial count and the initial contour area;
determining a count of background gene molecules within a preset region of the target region and a region area of the preset region;
determining a background gene molecule density based on the count of the background gene molecules and the region area; and
determining a cell to which the background gene molecules belong based on the gene molecule density and the background gene molecule density.

2. The gene image data correction method of claim 1, wherein determining a cell to which the background gene molecules belong based on the gene molecule density and the background gene molecule density comprises:
in response to a difference between the gene molecule density and the background gene molecule density being within a predetermined range, correcting the background gene molecules as gene molecules belonging to the target cell.

3. The gene image data correction method of claim 2, wherein determining a background gene molecule density based on the count of the background gene molecules and the predetermined area comprises:
determining a plurality of preset regions of different sizes; and
determining background gene molecule densities corresponding to the plurality of preset regions, respectively; and
correcting the background gene molecules as gene molecules belonging to the target cell comprises:
correcting background gene molecules within a preset region as gene molecules belonging to the target cell, where the background gene molecules within the preset region exhibit the smallest difference in gene molecule density compared to the target cell.

4. The gene image data correction method of claim 1, wherein determining an initial count of gene molecules belonging to a target cell in a gene image comprises:
acquiring a microscope image and a gene image of a biological sample;
performing image registration on the microscope image and the gene image of the biological sample;
performing cell segmentation on the microscope image to obtain a cell segmentation result;
determining a gene image of a target cell based on the cell segmentation result and an image registration result of the gene image; and
determining an initial count of gene molecules belonging to the target cell based on the gene image of the target cell.

5. The gene image data correction method of claim 4, wherein determining an initial contour area of a target region occupied by the gene molecules comprises:
calculating, by using a convex hull algorithm, an initial contour area of a target region occupied by the gene molecules based on the cell segmentation result.

6. The gene image data correction method of claim 1, wherein determining a gene molecule density of the target cell based on the initial count and the initial contour area comprises:
calculating a ratio of the initial count to the initial contour area to determine the gene molecule density of the target cell.

7. The gene image data correction method of claim 1, wherein determining a background gene molecule density based on the count of the background gene molecules and the region area includes:
calculating a ratio of the molecule of the background gene molecules to the region area of the background gene molecules to determine the background gene molecule density.

8. The gene image data correction method of claim 1, wherein after determining a cell to which the background gene molecules belong based on the gene molecule density and the background gene molecule density, the method further comprises:
generating gene expression information of the target cell based on a corrected result of gene molecules belonging to the target cell.

9. A gene image data correction system, comprising:
at least one processor, comprising:
a determination module configured to determine an initial count of gene molecules belonging to a target cell in a gene image and an initial contour area of a target region occupied by the gene molecules; determine a gene molecule density of the target cell based on the initial count and the initial contour area; determine a count of background gene molecules within a preset region of the target region and a region area of the preset region; and determine a background gene molecule density based on the count of the background gene molecules and the region area; and
a correction module configured to determine a cell to which the background gene molecules belong based on the gene molecule density and the background gene molecule density.

10. An electronic device, comprising a memory, at least one processor, and a computer program stored in the memory and executable by the at least one processor, wherein the computer program, when executed by the at least one processor, causes the at least one processor to perform the gene image data correction method of claim 1.

11. A non-transitory computer-readable medium storing computer instructions that, when executed by at least one processor, cause the at least one processor to perform the gene image data correction method of claim 1.
